(19) United States
(12) Patent Application Publication
(10) Pub. No.: US 2022/0054807 A1
(43) Pub. Date: Feb. 24, 2022

(54) CATHETER PUMP DEVICE FOR LOCAL REDUCTION OF VENOUS PRESSURE

(71) Applicant: 3rdCirc LLC, San Diego, CA (US)

(72) Inventor: George Itkin, Moscow (RU)

(73) Assignee: 3rdCirc LLC, San Diego, CA (US)

(21) Appl. No.: 17/410,056

(22) Filed: Aug. 24, 2021

Related U.S. Application Data
(60) Provisional application No. 63/069,248, filed on Aug. 24, 2020.

(51) Int. Cl. A61M 27/00; A61M 39/24; A61M 25/00; A61M 25/10

(52) U.S. Cl. CPC A61M 27/002; A61M 39/24; A61M 25/007; A61M 2202/0405; A61M 2210/12; A61M 2039/2406; A61M 2039/244; A61M 25/10

(57) ABSTRACT

Various catheter systems using a patient's own bodily fluids to increase local fluid velocity and therefore localized lower pressure via the Venturi effect. Exemplary systems utilize a pump to suction and eject blood to/from an attached catheter. One catheter comprises a valve moveable such that the area of the opening at the distal end for fluid passage is larger during suction than during ejection. Another catheter comprises openings in the sidewall and a balloon at the distal end to temporarily occlude the vessel during suction. Another catheter comprises a narrow distal opening and multiple openings with valves in the catheter sidewall. When catheters are deployed near a lymphatic opening into the circulation, such as at the thoracic duct, the ejection of blood from the catheter may cause a localized pressure reduction aiding in the reintroduction of lymph into the circulation.

### PRIORITY

The present patent application is related to, and claims the priority benefit of, U.S Provisional Patent Application Ser. No. 63/069,248, filed on Aug. 24, 2020, the contents of which are hereby incorporated by reference in their entirety into this disclosure.

### BACKGROUND

The lymphatic system is known as the third circulatory system, having an extensive network of distensible channels that parallels the vascular systems and that drains into the veins. The lymph circulation collects and transports excess tissue fluid and extravasated plasma protein, absorbed lipids, and other large molecules from the intestinal space back to the venous system (jugular and subclavian veins) via the thoracic duct (TD). In particular, and under normophysiologic conditions, the thoracic duct drains into the left subclavian vein, and the right lymphatic duct drains into the right subclavian vein. However, under pathologic conditions, there may be an outflow obstruction, constriction, or congestion. This congestion may be anatomic or restrictive in regard to increased outflow resistance due to high lymphatic drainage in the presence of, for example, congestive heart failure (CHF) or other venous insufficiencies

In normal mammals, it is estimated that 40% of the total plasma protein pool and an equivalent fluid to the total plasma volume are returned to the blood through the TD each day at approximately 1 ml/min. Unlike the arterial and venous counterparts, the lymphatic system is much less characterized and hence provides enormous opportunities for discovery of novel diagnostics and therapeutics.

There are both diagnostic and therapeutic targets for TD interventions which were pioneered by Dr. Cope two decades ago (Cope, 1995; Cope et al, 1997). For the former, changes in flow pressure and composition of TD can aid differential diagnosis of various disorders such as metastatic cancer, intestinal tuberculosis, Whipple disease, hepatic cirrhosis, bacterial infections, parasites, fungi, etc. to name just a few. On the latter, there are three major classes of therapy via TD access: 1) Removal of excess fluid or decompression of lymphatic system, 2) Elimination of toxic substance dissolved in lymph, and 3) Depletion of cells circulating in the TD.

In view of the foregoing, the present disclosure includes disclosure to address the therapeutic targets, namely the decongestion of the lymphatic system by reducing venous pressure, so to treat CHF and other disorders relating to the lymphatic system.

In acute or congestive heart failure conditions, the right heart pressures are elevated, as is the pressure at the subclavian vein, which is where lymph drains from the thoracic duct. Under these conditions, the lymph flow from the thoracic duct is reduced (due to the higher pressure in the subclavian), which causes undesirable congestion of lymph at the veno-lymph junction (i.e.,of the lymphatic system). Specifically, the higher pressure. in the subclavian vein, causes increased lymph formation (primarily by the liver) and this lymph then flows into the thoracic duct, which carries the lymph toward the subclavian vein. However, the increased pressure in the subclavian vein (during heart failure) impedes the drainage/flow of lymph and results in localized lymph congestion, with the associated signs and symptoms, such as undesirable fluid retention leading to ascites in the abdomen, fluid accumulation in the pericardial sac surrounding the heart, renal failure,and pulmonary edema, for example.

Currently, treatment to relieve congestion of the lymphatic system is accomplished using pharmaceuticals, such as diuretics and/or vasodilators. For more advanced heart disease conditions, current treatments may include supplemental oxygen to assist in breathing, or hospitalization for invasive procedures to actively drain excess fluid from the body. It would certainly be desirable to improve treatment methods and relieve the undesirable symptoms of lymphatic congestion for patients.

Disclosed herein are devices, methods, and systems that locally reduce the pressure at the veno-lymph junction (i.e., the junction of the subclavian/central vein and the thoracic duct) to increase the thoracic duct lymph flow. A physical principle by which this local pressure reduction may be accomplished is known as the Venturi effect (which stems from Bernoulli's principle of conservation of energy). Disclosed herein are devices, methods, and systems which accomplish a Venturi effect (i.e., increasing flow velocity to decrease pressure) near the veno-lymph junction to enhance lymph drainage into the venous subclavian vein/circulation in particularly acute conditions. It would further be desirable to treat patients using minimally invasive devices, methods, and systems which alter blood flow conditions in situ, while minimizing the amount of blood removed from the patient's body. The minimally invasive devices, methods, and systems disclosed herein create the advantageous blood flow conditions to relieve lymph congestion in situ, thus improving the standard of care and patient recovery rates, while minimizing adverse risks to the patient during a procedure.

### BRIEF SUMMARY

In one embodiment a device for reducing pressure at a veno-lymph junction to alleviate lymphatic congestion, comprises: a catheter and at least one opening at or near a distal end of the catheter having an effective area allowing the passage of fluid therethrough; and a valve having an open position and a closed position, wherein the valve at least partially blocks the at least one opening in the closed position; wherein the valve is configured to enter the closed position in response to positive pressure within the catheter and further configured to enter an open position in response to negative pressure within the catheter; wherein when the valve is in the closed position, the effective area of the at least one opening is smaller than when the valve is in the open position.

In a further embodiment, the valve defines a small opening for the passage of fluid therethrough when the valve is in the closed position. In a further embodiment, the valve comprises two semi-circular flaps pivotally coupled to the catheter wall at or near the center of the catheter such that in the closed position the two semi-circular flaps pivot into a position generally perpendicular to a longitudinal axis of the catheter. In a further embodiment, the small opening is defined by both of the two semi-circular flaps in the closed position. In a further embodiment, the small opening is elongated to optimize the blood flow velocity therefrom.

In another embodiment a device for reducing pressure at aveno-lymph junction to alleviate lymphatic congestion, comprises: a catheter and at least one opening at or near a distal end of the catheter having an effective area allowing the passage of fluid therethrough; and a valve having an open position and a closed position, wherein the valve at least partially blocks the at least one opening in the closed position; wherein the valve is configured to enter the closed position in response to positive pressure within the catheter and further configured to enter an open position in response to negative pressure within the catheter; wherein when the valve is in the closed position, the effective area of the at least one opening is smaller than when the valve is in the open position, wherein the valve comprises two semi-circular flaps pivotally coupled to the catheter wall at or near the center of the catheter such that in the closed position the two semi-circular flaps pivot into a position generally perpendicular to a longitudinal axis of the catheter.

In a further embodiment, the device further comprises a protrusion or rim at the distal end of the catheter wherein the rim or protrusion limits travel of the flaps. In another embodiment, the flaps are oversized so as to rest along the catheter wall when in the closed position

In one embodiment a device for reducing pressure at a veno-lymph junction to alleviate lymphatic congestion, comprises: a catheter and at least one opening at or near a distal end of the catheter having an effective area allowing the passage of fluid therethrough; and a valve having an open position and a closed position, wherein the valve at least partially blocks the at least one opening in the closed position; wherein the valve is configured to enter the closed position in response to positive pressure within the catheter and further configured to enter an open position in response to negative pressure within the catheter; wherein when the valve is in the closed position, the effective area of the at least one opening is smaller than when the valve is in the open position, wherein the at least one opening further comprises a second opening wherein the second opening is valveless and disposed at the distal tip of the catheter.

In one embodiment a device for reducing pressure at a veno-lymph junction to alleviate lymphatic congestion. comprises: a catheter and at least one opening at or near a distal end of the catheter having an effective area allowing the passage of fluid therethrough; and a valve having an open position and a closed position, wherein the valve at least partially blocks the at least one opening in the closed position; wherein the valve is configured to enter the closed position in response to positive pressure within the catheter and further configured to enter an open position in response to negative pressure within the catheter; wherein when the valve is in the closed position, the effective area of the at least one opening is smaller than when the valve is in the open position; wherein the at least one opening further comprises a second opening wherein the second opening is valveless and disposed at the distal tip of the catheter; wherein the at least one opening further comprises four openings disposed on the catheter wall, and wherein the valve comprises four valves configured to block the four openings.

In one embodiment a device for reducing pressure at a veno-lymph junction to alleviate lymphatic congestion comprises: a catheter; a balloon disposed on or near the distal end of the catheter; and at least one opening disposed proximal to the balloon.

In a further embodiment, the at least one opening comprises three openings.

In a further embodiment, the balloon comprises an inflation medium and the inflation medium is a patient's own bodily fluid.

In a method for reducing pressure at a veno-lymph junction to relieve lymphatic congestion at a thoracic duct, the method comprises the steps of: positioning a catheter having an opening at or near a distal end of the catheter within a subclavian vein such that the opening is at or near the veno-lymph junction; suctioning a fluid present in the subclavian vein through the opening of the catheter creating an area of lowered pressure in the subclavian vein near the veno-lymph junction; and ejecting the suctioned fluid through the distal end of the catheter such that a velocity of the ejected fluid is higher than a velocity of fluid normally present within the vein thereby creating a localized Venturi effect.

In a further embodiment the step of ejecting the suctioned fluid is performed for duration longer than the step of suctioning the fluid.

In a further embodiment the step of ejecting the suctioned fluid further comprises the steps of: closing a valve such that the opening is at least partially blocked by the valve; and ejecting the suctioned fluid though a smaller opening defined within the valve when the valve is in a closed position.

In a further embodiment the step of ejecting the suctioned fluid further comprises the steps of: closing a valve such that the opening is blocked by the valve; and ejecting the suctioned fluid through a second opening on the distal tip of the catheter.

In a further embodiment the opening further comprises three openings and wherein the step of positioning a catheter having an opening at or near its distal end within a subclavian vein such that the opening is at or near the veno-lymph junction, further comprises the step of positioning the three openings such that they extend both proximal to and distal to the thoracic duct in the subclavian vein.

In a further embodiment, the step of suctioning fluid is performed while a balloon positioned on the distal end of the catheter and distal to the openings is inflated, and the step of ejecting suctioned fluid is performed while the balloon is deflated. In a further embodiment the step of suctioning fluid further comprises the step of introducing the suctioned fluid into the balloon, and the step of ejecting the suctioned fluid further comprises the step of passing the fluid in the balloon

In a further embodiment for a method of reducing pressure at a veno-lymph junction to relieve lymphatic congestion at a thoracic duct, the method further comprises the step of synchronizing a pump to suction and eject fluid with the contraction of the thoracic duct.

### BRIEF DESCRIPTION OF THE DRAWINGS

The disclosed embodiments and other features, advantages, and disclosures contained herein, and the matter of attaining them, will become apparent and the present disclosure will be better understood by reference to the following description of various exemplary embodiments of the present disclosure taken in conjunction with the accompanying drawings, wherein:

FIG. 1A illustrates a perspective view of a distal end of a catheter having a flow restricting valve in the open position;

FIG. 1B illustrates a perspective view of a distal end of a catheter having a flow restricting valve in the closed position with a small hole therein;

FIG. 1C illustrates an exemplary distal end view of a catheter having a flow restricting valve in the open position;

FIG. 2A illustrates a side cross-sectional view of the distal end of a catheter having a flow restricting valve in the open position;

FIG. 2B illustrates a side cross-sectional view of the distal end of a catheter having a flow restricting valve in the closed position with a small hole therein;

FIG. 3A illustrates a side cross-sectional view of the veno-lymph junction having a distal end of an exemplary balloon catheter positioned therein with the balloon inflated;

FIG. 3B illustrates a side cross-sectional view of the veno-lymph junction having a distal end of an exemplary balloon catheter positioned therein with the balloon deflated;

FIG. 4 depicts a graph depicting the relative states of the pump and balloon during inflation and deflation;

FIG. 5A illustrates a side cross-sectional view of the distal end of a catheter having a holes or vents therein in the closed position;

FIG. 5B illustrates a side cross-sectional view of the distal end of the catheter having holes or vents therein in an open, or partially open, position; and

FIG. 6 depicts a catheter device of the present invention having sensors installed in various positions on and within the catheter.

An overview of the features, functions and/or configurations of the components depicted in the various figures will now be presented. It should be appreciated that not all of the features of the components of the figures are necessarily described. Some of these non-discussed features, such as various couplers, etc., as well as discussed features are inherent from the figures themselves. Other non-discussed features may be inherent in component geometry and/or configuration.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

The present disclosure includes disclosure relating to the first therapeutic class (i.e., decongestion of lymphatic system) with application to congestive heart failure (CHF) and other disorders.

The feasibility of TD lymph decompression/drainage has already been demonstrated in patients five decades ago (Dumont et al, 1963; Witte et al, 1969). Thoracic duct cannulation was made surgically in CHF patients (a total of 17 patients in two studies, mostly class IV stage) to allow drainage of the distended TD. The decompression therapy provided immediate resolution of a number of signs and symptoms, including significant reductions of the following: venous pressure, distention of veins, and peripheral edema. Ascites and hepatomegaly also diminished or resolved completely in those patients.

Despite the tremendous efficacy of this approach and relative safety, there are two major shortcomings, namely 1) required surgical access of TD, and 2) it only provides temporary relief as it does not address the root cause of lymphatic congestion. To reap a chronic therapeutic benefit, for example, the procedure must be repeated frequently. The first shortcoming has been addressed given the present non-surgical (percutaneous) access of the TD; however, a solution to the second shortcoming has previously not been addressed. The present disclosure addresses this second shortcoming, namely to provide a chronic therapeutic benefit previously unknown and unavailable in the medical arts.

To address this second shortcoming, a major question is what constitutes the bottleneck to drainage of lymphatic fluid into the venous system to avoid congestion/edema/ascites in CHF in the first place. This question can only be answered if we have an intimate understanding of the major determinants of lymphatic flow; namely: 1) resistance of lymphatic channels, and 2) the pressure gradient across the lymphatics. The former is dictated by the architecture (morphometry, branching pattern, etc.) and mechanical properties (passive compliance, active smooth muscle contraction, distribution of lymphatic valves, etc.) of the lymphatic system in health and in CHF. The latter requires an understanding of the hemodynamic conditions (pressure difference) between the lymphatic terminals and drainage veins.

Such an understanding has allowed us to design solutions for decompression of the lymphatic system as included in the present disclosure. Specifically, creation of devices, systems, and methods for locally accelerating blood flow velocity (to decrease pressure as per the Venturi effect) and thus facilitate drainage of lymph from the thoracic duct, can address the second shortcoming noted above. As drainage of the lymphatic system to the venous system is critical (when dictated by a pressure gradient), such devices, systems, and methods as referenced in further detail herein, can provide the acute and chronic relief needed to maintain a decongested lymphatic system.

The basic premise is that an elevated systemic venous pressure in CHF reduces the pressure gradient for lymphatic flow and a connection to a lower pressure venous system can increase/restore the pressure gradient. The requirements of any device, system, and method (diameter, lengths, opening/closing pressures, etc.), lymphatic and venous locations (e.g., TD-to-pulmonary vein given the lower pressure than the systemic veins where drainage normally occurs, Cole et al, 1967), etc., could only be determined rationally once the above noted characterization of the lymphatic system are made.

Described herein are devices, methods, and systems that locally reduce the pressure at the veno-lymph junction or anastomosis (i.e., the junction of the subclavian/central vein and the thoracic duct) to increase lymph flow or drainage out of the thoracic duct, utilizing the Venturi effect. The Venturi effect is accomplished through a local increase in the velocity of the blood flow, which then decreases the pressure, to conserve total energy according to Bernoulli's principle. A catheter may generally referred to herein as a catheter, device, or catheter device, and any combination of a catheter, balloon, valve, and/or pump, may generally be referred to herein as a system 200. Additionally, while the description of the embodiments may refer to blood or fluid, the two are used interchangeably to refer generally to the liquid within the vessels and ducts described; which may be lymph, blood, indicators such as contrast dye or other material introduced into the body as described herein, or any combination of the preceding.

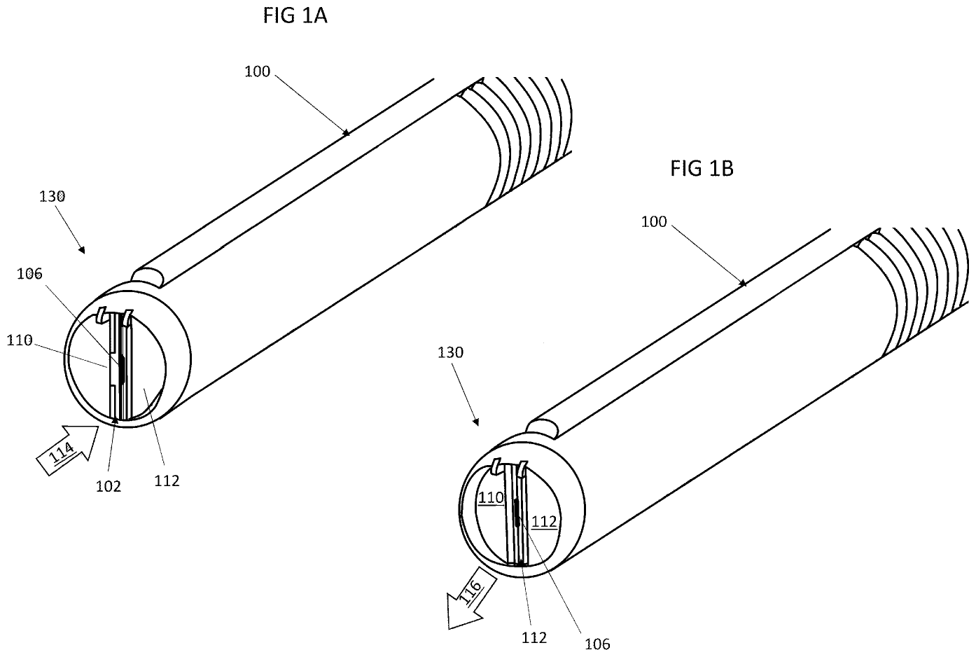

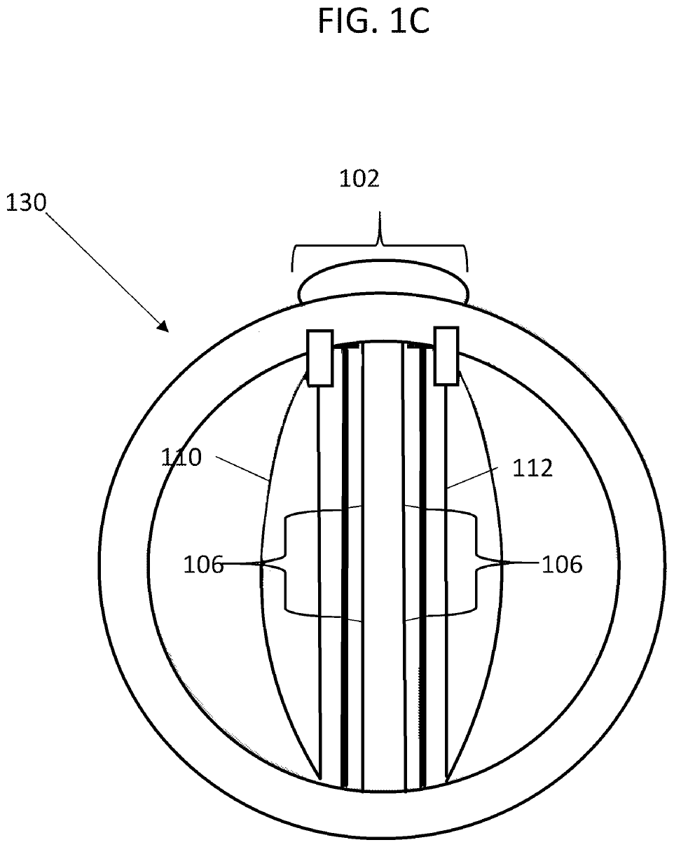

FIGS. 1A and 1B show the distal end 130 of an exemplary catheter device 100 of the present disclosure. As shown therein, catheter 100 may comprise a narrowing of its distal end 130, or valve 102, configured to forcefully eject blood back into the vein, resulting in the Venturi effect, thus creating an area of low pressure at or near the veno-lymph junction to draw lymph out of the thoracic duct and alleviate lymphatic congestion.

The valve 102 may be a flow restriction valve to allow blood/fluid to flow into, or be suctioned into, the distal end 130 of the catheter 100 when in the open position, as depicted in FIG. 1A. The valve 102 may also be closed, as shown in FIG. 1B, to stop blood/fluid from flowing into the catheter 100, and to narrow the distal end 130 to only the small opening 106, to more forcefully eject or expel blood out of the catheter 100 (i.e., at a higher velocity). The suction and ejection of blood may be accomplished using a pump, operably coupled to the proximal end of the catheter 100. The pump may be an external pump (not shown), such as electro-mechanical unit or an electro-pneumatic unit, for example. In one embodiment, the catheter 100 and pump may operate as a system 200, using periodic rapid removal of blood from the subclavian, and an ejection of blood out of catheter 100 from small opening 106, to create a Venturi effect at the area in the subclavian vein close to the thoracic duct (i.e., the veno-lymph junction).

In a first embodiment, a catheter 100 may be inserted through the jugular vein, into the region of the subclavian vein close to the thoracic duct, where the lymph flow enters the venous system. Catheter 100 may also be inserted through the jugular vein, brachial vein, subclavian vein percutaneously or surgically, as is known in the art.

In one exemplary embodiment, catheter 100 may have a flow restricting valve 102 thereon, as shown in FIGS. 1A, 1B and 1C. As best shown in end-view FIG. 1C (of distal end 130 of catheter 100, the flow restricting valve 102 may be generally formed of two semi-circle shaped flaps 110, 112 pivotally coupled to the luminal wall of the catheter 100 at or ear the center (or largest diameter section) of the catheter 100. The two flaps 110 112 of the flow restricting valve 102 may operate by pivoting toward and away from one another (to open and close) similar to a butterfly hinge. The two flaps 110, 112 and/or an area between the two flaps 110, 112, may further comprise smaller semi-circular cut-outs (best shown in FIG. 1C) which, together, form a small opening 106 when the valve 102 is in the closed position.

The two flaps 110, 112 of the flow restricting valve 102 may operate by pivoting into the lumen at the distal end 130 of the catheter 100, when in the open position (shown in FIGS. 1A and 1C). When the flow restricting valve 102 is in the closed position (shown in FIG. 1B), the two flaps 110, 112 may pivot back into a position generally perpendicular to the longitudinal axis of the catheter 100 to block flow, or a majority of flow, back into the catheter 100. When the flow restricting valve 102 is in the closed position, a small opening or hole 106 may remain, or be formed, in the center of the flow restricting valve 102. As shown in FIG. 1B, the small opening or hole 106 may have a slighted elongated or oval shape, to optimize the velocity and/or acceleration at which the blood is ejected therefrom, to further optimize the effectiveness of the Venturi effect (based upon Bernoulli's principle). However, it should be understood that the flow restricting valve 102 and small opening or hole 106 may have a variety of different shapes and configurations, and similarly, the method of opening and closing the flow restriction valve 102 may be done in a number of different ways, all of which are considered to be within the scope of the present disclosure.

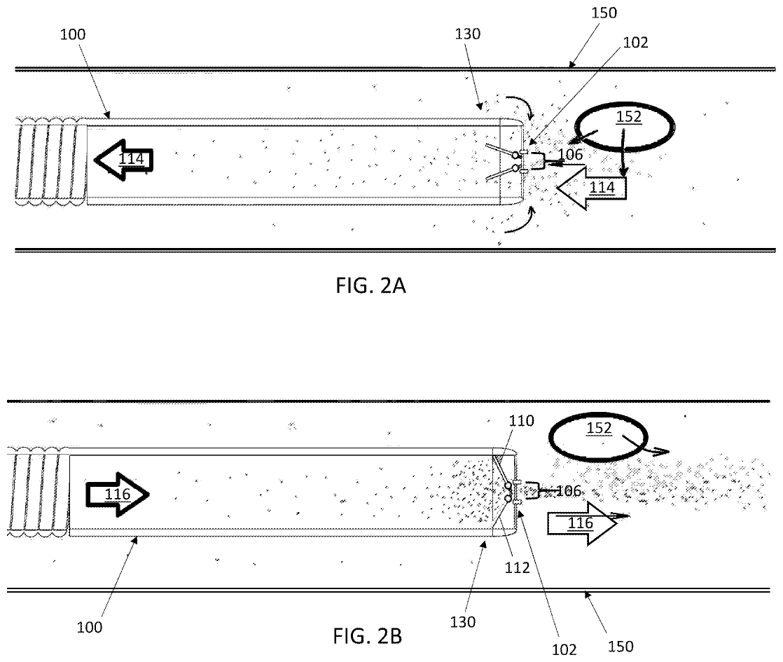

In the embodiment of FIGS. 1A-1C, the valve 102, and in particular the flaps 110, 112, moves from the open position to the closed position in response to the pump applying suction or pumping blood out of the catheter 100. When the pump is activated to suction the contents of the vessel and catheter, the suction force will draw open the flaps 110, 112 such as in FIG. 2A. When the pump is activated to eject blood, the flaps are pushed distally into a position perpendicular to the longitudinal axis of the catheter, partially blocking the lumen of the catheter, as shown in FIG. 2B. In this embodiment, the flaps move only under the power of the suction and ejection pressure applied from the pump. A protrusion or rim at the distal end 130 of the catheter may limit travel of the flaps, such that the flaps are in a maximum closed position once a threshold ejection pressure from the pump is reached or exceeded. In another embodiment, the flaps 110, 112 could he oversized so as to rest along the catheter wall when in the closed position. In this embodiment, the flaps may not extend fully perpendicular to the longitudinal axis in the closed position, but remain slightly less than perpendicular.

Additionally, FIGS. 2A and 2B show side cross-sectional views of the distal end 130 of the catheter 100 having the flow restricting valve 102 in both the open and closed positions within the subclavian vein 150. FIG. 2A show the flow restricting valve 102 in the open position and further illustrates the suction of blood into the distal end 130 (as shown by directional arrow 114). FIG. 2B shows the flow restricting valve 102 in the closed position, with the exception of a small opening or hole 106 disposed therein. FIG. 2B further illustrates the ejection of blood (as shown by directional arrow 116) out of the small opening or hole 106 in the distal end 130 of the catheter 100.

The acceleration or increase in velocity of blood flow (shown as arrows 114 or 116), accomplished by forcefully ejecting blood out of small opening or hole 106 (in direction of arrows 116) in the distal end 130 of catheter 100, causes a local decrease in pressure along with the suction effect (which helps draw the lymph out of the thoracic duct 152) to alleviate lymphatic congestion. Further, if the distal end 130 of catheter 100 is positioned close to the inflow point of the thoracic duct 152 (i.e., at the veno-lymph junction), it results in an area of lower pressure within the vein, to further facilitate the removal of lymph from the thoracic duct 152, according to the Verturi effect.

With the valves in the open position, the distal end 130 of the catheter comprises a much larger effective area that is accessible for fluid (such as blood and lymph) passage. In contrast, when the valves are in the closed position, the area accessible for fluid passage is much smaller, being limited to the small area 106. Where the area is larger, a larger volume of blood can he suctioned through the catheter and quickly, thereby allowing the rapid removal of blood. When the flaps are closed, the smaller area restricts blood passage. As a result, the ejection of suctioned bloods will occur for a duration longer than the suctioning of blood.

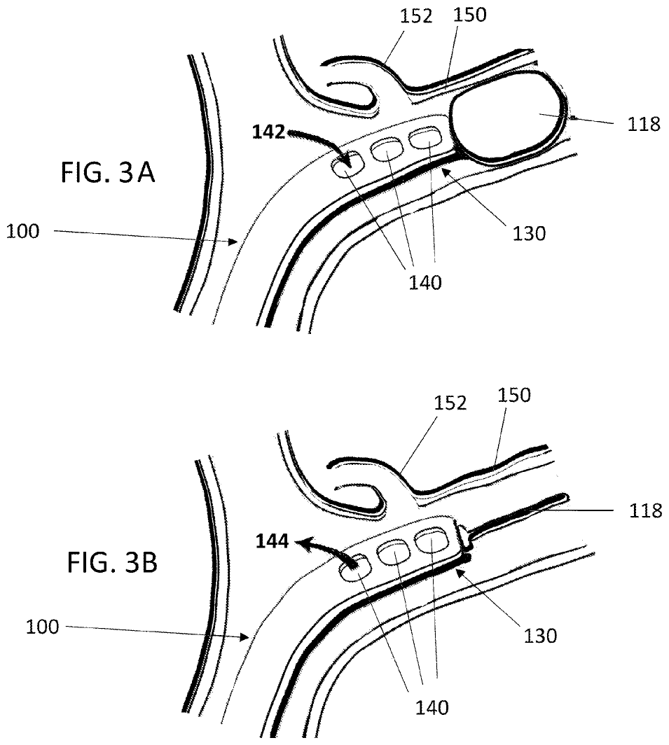

In another embodiment, shown in FIGS. 3A and 3B, to further increase the effect of reducing pressure within the subclavian vein 150 (via the Venturi effect) to alleviate lymphatic congestion, a balloon 118 may be positioned at or over the distal end 130 of catheter 100 and a number of openings 140 may also be disposed along the catheter's 100 length. As shown in FIG. 3A, the balloon 118 may be inflated within the subclavian vein 150, at or near the veno-lymph junction. In FIGS. 3A and 3B, the openings are positioned at the veno-lymph junction and extend across the junction. Additionally, the suctioning of blood flow back into (arrow 142) the catheter 100 through a number of openings 140 along the distal end 130 thereof, when positioned at or near the veno-lymph junction, may further reduce venous pressure at the veno-lymph junction to alleviate lymphatic congestion. Suction and ejection can he accomplished by an attached pump (not shown). FIG. 3A illustrates a cross-sectional view of the distal end 130 of catheter 100 with balloon 118 inflated within the subclavian vein 150, near the junction with the thoracic duct 152. FIG. 3B illustrates the distal end 130 of the catheter 100 in the same location, but with the balloon 118 deflated and flow being reversed to flow out of (shown generally as arrow 144) the openings 140 within catheter 100. In this embodiment, catheter contents are suctioned and ejected from the openings 140 in the sidewall of the catheter 100 rather than the distal end, as the balloon is positioned at the end. Alternate embodiments are envisioned where the catheter lumen could extend through the balloon to allow suction and ejection from the distal end.

In an exemplary operation, the catheter 100 having a balloon 118 on distal end 130 may be advanced into the subclavian vein 150 near or at the thoracic duct 152. Once in position, balloon 118 may be slowly inflated to occupy part of the venous vessel (i.e., subclavian vein 150), as shown in FIG. 3A. The balloon 118 may be inflated using gas, such as CO₂ or helium, or a fluid. Once the desired balloon 118 inflation has been achieved, then a pump coupled to catheter may quickly suction blood into the catheter 100 (shown generally by arrow 142), via openings 140, removing some blood out of the subclavian vein 150, which decreases the pressure in the subclavian vein 150 and thus facilitates the drainage or flow of lymph out of the thoracic duct 157.

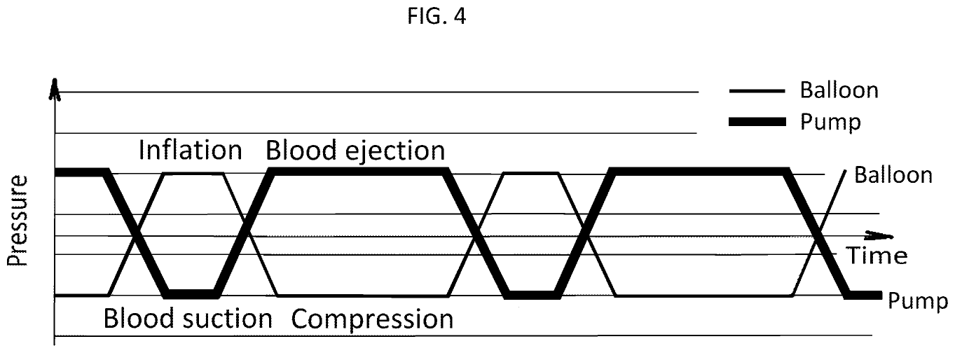

FIG. 4 shows the relative state of the balloon 118 and the pump during device operation. When the balloon is deflated (or compressed), blood can be ejected from the openings to increase the speed of local blood flow. During balloon inflation, suction at the openings can draw blood into the catheter and also help drawn lymph into the vein. Also as in the embodiments, FIG. 4 shows the blood suction and balloon inflation stage lasts for a shorter time duration than the blood ejection and balloon deflation/compressions stage. The two stages are alternated and can be repeated as desired.

Additionally, a single pump can be used to inflate/deflate the balloon and suction/eject blood. When the balloon is inflated, a corresponding suction can be induced in the catheter. That is, the suctioned blood can be introduced into the balloon. Then the balloon can be compressed to eject blood through the catheter and into the bloodstream. As such only a single pump is required suction/eject bodily fluid and inflate and deflate/compress the balloon. Furthermore, if the balloon bursts, the use of the patient's own blood as inflation medium eliminates the danger of introducing a toxic substance into the bloodstream

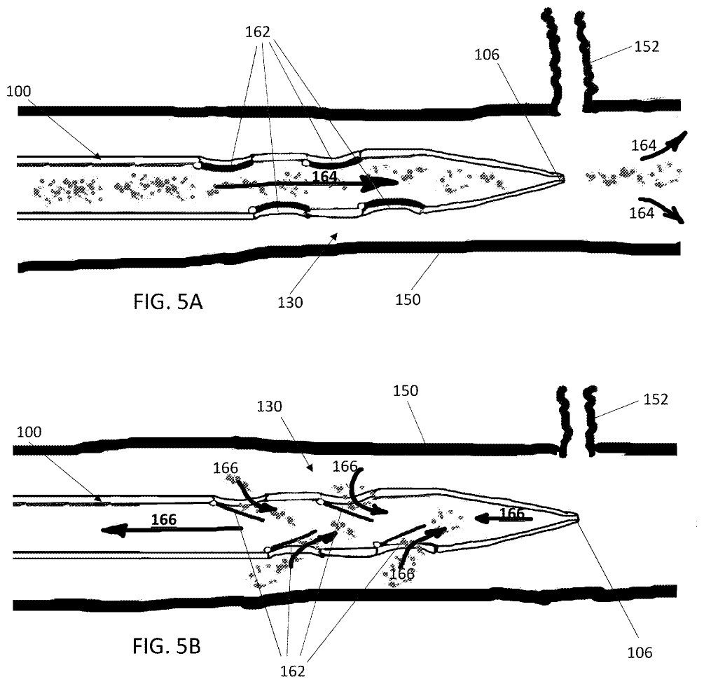

In another embodiment, shown in FIGS. 5A and 5B, to further increase the effect of reducing pressure within the subclavian vein 150 (via the Venturi effect) to alleviate lymphatic congestion, the distal end 130 of catheter 100 may be narrowed or pointed and may form a distal opening 106, as well as any number of openings and/or valves (shown generally as 162) disposed along the catheter's 100 length. In this embodiment, the openings 162 are disposed in the catheter sidewall. The distal opening may he valve-less, positioned on the distal tip of the catheter, and be smaller than the catheter lumen. As shown in FIG. 5A, the distal end 130 of the catheter 100 may be positioned within the subclavian vein 150, at or near the veno-lymph junction. With continuing reference to FIG. 5A, the openings and/or valves 162 along the length of the catheter 100 may be blocked and closed and flow may be activated and directed out of the catheter's 100 distal opening 106 (in the direction of arrows 164). As shown in FIG. 5B, the openings and/or valves 162, may then be opened such that the valves are not blocking the openings to allow flow back into the catheter 100 (shown by arrows 166) and/or suction may be activated to draw fluid back into the catheter 100 (shown by arrows 166) through the distal opening 106, as well as through openings and/or valves 162, to help reduce pressure. at the veno-lymph junction to further alleviate lymphatic congestion.

In one embodiment, catheter 100 may be operably coupled to a pump at its proximal end (not shown), forming a system 200. The pump may be an electro-mechanical or electro-pneumatic unit that provides the amplitude, duration of the suction (of blood into distal end 130 of catheter 100), and ejection or discharge phases (of blood out of the small opening 106), as specified by a control system. The pump may also operate to forcefully eject the blood out of the catheter 100 (in the direction of arrows 116, 144, and/or 164) and/or into the catheter (in the direction of allows 114, 142, and/or 166) using pneumatic pressure. As with the embodiment pictured in FIGS. 1A-2B, the openings 162 may comprise flaps that are responsive to suction and ejection pressure from the pump. Suction pressure draws open the flaps allowing more blood to enter the catheter lumen. Ejection pressure closes the flaps, ensuring blood is forcefully ejected from the distal end of the catheter, and thereby creating a lower local pressure in the area of the thoracic duct 152 outlet which assists in lymph drainage. Furthermore, the picture flaps are a hinged single piece, but other shapes or attachment points are envisioned.

In addition, like the embodiments of FIGS. 1A-2B, the effective area available for blood passage is smaller when the valves 162 are in the closed position (during blood ejection), as in FIG. 5A, than when the valves 162 are in the open position (blood suction), as in FIG. 5B. In the embodiment of FIGS. 5A-5B, the closed position leaves only the distal opening 106 available for blood passage and the open position allows blood to enter through the distal opening 106 and all valves 162. Also like the previous embodiments, the difference in area available for blood passage provides for a large volume of blood to be suctioned over a short duration of time through the large area, and for the suctioned blood to be ejected rapidly over a longer duration of time.

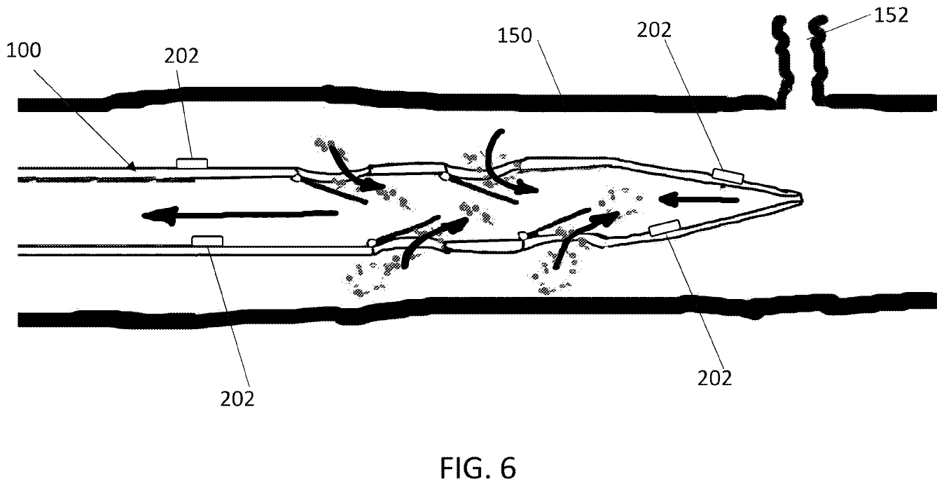

A variety of sensors 202 may be installed on or within the catheters 100 of the present disclosure and in varying positions along the length of the catheter 100 as pictured in FIG. 6 and described below. Sensor positions 202 as shown in FIG. 6 are exemplary and non-exhaustive. Furthermore, although the sensors 202 of 6 are illustrated using the embodiment of FIGS. 5A-5B the sensors 202 could be similarly positioned on any embodiment as described in the disclosure and as pictured in the figures.

The outlet portion of the catheter (such as any openings 106, 140, and/or 162 disposed therein) may further be equipped with a pressure sensor 202 to allow the operator, or the control system, to automatically optimize the operation of the pump. An exemplary embodiment is shown in FIG. 6.

In some embodiments, the cycle of the pump may be synchronized with contraction of the thoracic duct or lymphatic duet 152. In order to determine the frequency of thoracic duct 152 peristalsis, and its point of lymph drainage into the venous system 150, a special sensor 202 may be installed on the catheter 100 of above described embodiments.

Detection of lymph flow from the thoracic duct 152 may be performed using a number of different types of sensors 202, such as, for example, a pressure sensor, optical sensor, ultrasonic sensor, temperature sensor, chemical sensor, laser sensor, and/or nuclear medicine sensor. Multiple sensors 202 may also be used. The sensor 202 may be placed on or within the catheter 100 as desired, for detection of indicators that are indicative of lymph drainage.

For example in some embodiments, the frequency of contractions of the thoracic duct 152 may also be detected by a sensor 202 that detects electrical signals of the chest duct contraction. In this example, the sensors may obtain conductance measurement indicative of the lumen size wherein a smaller lumen size would indicate the contraction of the thoracic duct, and therefore lymph entering the vein. In this embodiment, such sensors could be electrodes installed on the exterior of the catheter.

In another embodiment, the drainage point of the thoracic duct 152 may be determined (using the previously listed sensors)when an indicator is delivered to the lymphatic system by injection into the lymph nodes. Injections into the lymph nodes, vessels, or injections into the subcutaneous tissue may include indicators: optical or fluoroscopic dye, cold or hot solutions, ultrasonic contrast, indicators of nuclear medicine, radiographic dyes, chemical solutions, etc. This sensor 202 would collect information indicative of the contraction of the lymphatic duct and communicate this information such that the pump would base ejection and suction stages on the contraction stages of the ducts. The sensor could be installed exterior to the catheter to detect indictors flowing past, or the sensor could be installed within the catheter to detect indicators that are suctioned into the catheter lumen.

While various embodiments of devices, methods, and systems for relieving lymphatic congestion have been described in considerable detail herein, the embodiments are merely offered as non-limiting examples of the disclosure described herein. It will therefore be understood that various changes and modifications may be made, and equivalents may be substituted for elements thereof, without departing from the scope of the present disclosure. The present disclosure is not intended to be exhaustive or limiting with respect to the content thereof.

Further, in describing representative embodiments, the present disclosure may have presented a method and/or a process as a particular sequence of steps. However, to the extent that the method or process does not rely on the particular order of steps set forth therein, the method or process should not be limited to the particular sequence of steps described, as other sequences of steps may be possible. Therefore, the particular order of the steps disclosed herein should not be construed as limitations of the present disclosure. In addition, disclosure directed to a method and/or process should not be limited to the performance of their steps in the order written. Such sequences may be varied and still remain within the scope of the present disclosure.

### REFERENCES

Cope C. Percutaneous thoracic duct cannulation: feasibility study in swine. J Vasc Intery Radiol. 6(4):559-64, 1995.
Cope C, Timms I, Pavcnik D. Percutaneous transthoracic duct catheterization to the neck and esophagus: a feasibility study. J Vasc Intery Radiol. 8(5): 845-9, 1997.
DUMONT A E, CLAUSS R H, REED G E, TICE D A. LYMPH DRAINAGE IN PATIENTS WITH CONGESTIVE HEART FAILURE. COMPARISON WITH FINDINGS IN HEPATIC CIRRHOSIS. N Engl J Med. 269:949-52, 1963.
Witte M H, Dumont A E, Clauss R H, Rader B, Levine N, Breed E S. Lymph circulation in congestive heart failure: effect of external thoracic duct drainage. Circulation. 39(6):723-33, 1969.
Cole W R, Witte M H, Kash S L, Rodger M, Bleisch W R, Muelheims G H. Thoracic duct-to-pulmonary vein shunt in the treatment of experimental right heart failure. Circulation. 36(4): 539-43, 1967.

1. A device for reducing pressure at a veno-lymph junction to alleviate lymphatic congestion, comprising:
a catheter and at least one opening at or near a distal end of the catheter having an effective area allowing the passage of fluid therethrough; and
a valve having an open position and a closed position, wherein the valve at least partially blocks the at least one opening in the closed position;
wherein the valve is configured to enter the closed position in response to positive pressure within the catheter and further configured to enter an open position in response to negative pressure within the catheter;
wherein when the valve is in the closed position, the effective area of the at least one opening is smaller than when the valve is in the open position.

2. The device of claim 1, wherein the valve defines a small opening for the passage of fluid therethrough when the valve is in the closed position

3. The device of claim 2, wherein the valve comprises two semi-circular flaps pivotally coupled to the catheter wall at or near the center of the catheter such that in the closed position the two semi-circular flaps pivot into a position generally perpendicular a longitudinal axis of the catheter.

4. The device of claim 3, wherein the small opening is defined by both of the two semi-circular flaps in the closed position.

5. The device of claim 4, wherein the small opening is elongated to optimize the blood flow velocity therefrom.

6. The device of claim 3, further comprising a protrusion or rim at the distal end of the catheter wherein the rim or protrusion limits travel of the flaps.

7. The device of claim 3, wherein the flaps are oversized so as to rest along the catheter wall when in the closed position

8. The device of claim 1, wherein the at least one opening further comprises a second opening wherein the second opening is valveless and disposed at the distal tip of the catheter.

9. The device of claim 8, wherein the at least one opening further comprises four openings disposed on the catheter wall, and wherein the valve comprises four valves configured to block the four openings.

10. A device for reducing pressure at a veno-lymph junction to alleviate lymphatic congestion, comprising:
a catheter;
a balloon disposed on or near the distal end of the catheter; and
at least one opening disposed proximal to the balloon.

11. The device of claim 10, wherein the at least one opening comprises three openings.

12. The device of claim 10, wherein the balloon comprises an inflation medium and the inflation medium is a patient's own bodily fluid.

13. A method for reducing pressure at a veno-lymph junction to relieve lymphatic congestion at a thoracic duct, comprising the steps of:
positioning a catheter having an opening at or near a distal end of the catheter within a subclavian vein such that the opening is at or near the veno-lymph junction;
suctioning a fluid present in the subclavian vein through the opening of the catheter creating an area of lowered pressure in the subclavian vein near the veno-lymph junction; and
ejecting the suctioned fluid through the distal end of the catheter such that a velocity of the ejected fluid is higher than a velocity of fluid normally present within the vein thereby creating a localized Venturi effect.

14. The method of claim 13, wherein the step of ejecting the suctioned fluid is performed for duration longer than the step of suctioning the fluid.

15. The method of claim 13, wherein the step of ejecting the suctioned fluid further comprises the steps of:
closing a valve such that the opening is at least partially blocked by the valve; and
ejecting the suctioned fluid though a smaller opening defined within the valve when the valve is in a closed position.

16. The method of claim 14, wherein the step of ejecting the suctioned fluid further comprises the steps of:
closing a valve such that the opening is blocked by the valve; and
ejecting the suctioned fluid through a second opening on the distal tip of the catheter.

17. The method of claim 13, wherein the opening further comprises three openings and wherein the step of positioning a catheter having an opening at or near its distal end within a subclavian vein such that the opening is at or near the veno-lymph junction, further comprises the step of positioning the three openings such that they extend both proximal to and distal to the thoracic duct in the subclavian vein.

18. The method of claim 13, wherein the step of suctioning fluid is performed while a balloon positioned on the distal end of the catheter and distal to the openings is inflated, and the step of ejecting suctioned fluid is performed while the balloon is deflated.

19. The method of claim 18, wherein the step of suctioning fluid further comprises the step of introducing the suctioned fluid into the balloon, and the step of ejecting the suctioned fluid further comprises the step of passing the fluid in the balloon

20. The method of claim 18, further comprising the step of synchronizing a pump to suction and eject fluid with the contraction of the thoracic duct.
